(19) United States
(12) Patent Application Publication
(10) Pub. No.: US 2004/0153005 A1
(43) Pub. Date: Aug. 5, 2004

(54) Bone marrow aspiration device with curved tip

(76) Inventor: John A. Krueger, Milwaukee, WI (US)

Correspondence Address: Kimberly C. Diliberti, Allegiance Corporation, 1430 Waukegan Road, McGaw Park, IL, 60085, US

(21) Appl. No.: 10/037,795

(22) Filed: Jan. 3, 2002

(51) Int. Cl. A61B 10/00

(52) U.S. Cl. 600/571

(57) ABSTRACT

The invention disclosed herein relates to a biopsy aspiration device comprising an elongated cannula body having a proximal end, a distal tip and a linear longitudinal axis; a lumen running longitudinally through the interior of said cannula body and terminating at a proximal opening and terminating at a single laterally oriented distal opening; wherein said distal tip of said cannula body comprises an arcuate curved surface on the side opposite side to said laterally oriented distal opening, said arcuate curved surface terminating at said distal opening. The invention also includes a biopsy system including an outer cannula through which the aspiration device can operate. The aspiration device of the invention permits 360° rotational sampling of bone marrow.

### FIELD OF THE INVENTION

This invention relates to the field of medical devices useful for bone biopsy procedures. In particular, the invention pertains to a bone marrow aspiration device.

### BACKGROUND OF THE INVENTION

In the medical field, certain bone diseases and disorders sometimes require the collection of bone biopsy samples for diagnostic and treatment purposes. Biopsy devices known in the art typically contain a cannula having a sharpened edge and trocar or stylet residing within to aid in penetrating the cortex of the bone. Once inserted through the cortex and positioned within the softer bone, the trocar or stylet is removed and the cannula is further inserted to obtain a core sample.

Devices which obtain both a marrow sample and bone core sample have been developed. Joishy, U.S. Pat. No. 5,012,818 describes a dual channel cannula, wherein a marrow sample is aspired through one channel and a core sample obtained using the other. Because of its relatively large diameter, the device can create unnecessary discomfort to the patient. Another device described by Turkel et al. U.S. Pat. No. 5,257,632 is drawn to a bone marrow biopsy needle which can obtain a marrow sample and core sample in a single procedure. This device, however, obtains a core sample smaller in diameter than the aspiration cannula. Furthermore, the core sample is obtained prior to the aspired marrow sample, thereby obtaining an aspired marrow sample from the damaged region of the bone core sample.

There exists a need in the medical field for bone biopsy devices and systems which minimize patient discomfort and exhibit improved operability and efficiency. In particular, there is a need for bone biopsy devices which can obtain an aspired marrow sample and bone core sample from a single sampling site without significantly compromising sample integrity. There is also a need for an aspiration cannula which can reduce the likelihood of becoming clogged with tissue during its use.

### SUMMARY OF THE INVENTION

The invention provides a biopsy aspiration device adapted to obtain a bone marrow sample utilizing improved sampling technology. It has been discovered that a biopsy aspiration device can be constructed to permit use within an outer cannula, to obtain a bone marrow sample while minimizing adulteration of a bone core sample to be removed from the same site, to reduce or avoid obstruction from distally located tissue through which it is penetrated, and to ensure adequate marrow sampling by permitting 360° rotational sampling capability within the sampling site. The biopsy aspiration device of the invention can be used as part of a bone biopsy system comprising an outer cannula, such as a biopsy coring cannula, with a handle portion coupled to the outer cannula. The device of the invention is particularly useful in obtaining bone marrow samples in regions of a bone or in types of bones where obtaining adequate marrow samples is difficult. The device is especially useful in bone biopsy procedures wherein a marrow sample and core sample are both obtained from a single sampling site, since the biopsy aspiration device can collect a marrow sample without significant adulteration of the bone core sample to be obtained subsequently.

The invention provides a biopsy aspiration device adapted to obtain a bone marrow sample comprising:

an elongated cannula body having a proximal end and a distal tip and a linear longitudinal axis;

a lumen running longitudinally through the interior of said cannula body, said lumen terminating at a proximal opening and terminating at a single laterally oriented distal opening immediately adjacent to the distal tip;

wherein said distal tip of said cannula body comprises an arcuate curved surface on the side opposite to said laterally oriented distal opening, said arcuate curved surface terminating at said distal opening.

In a further embodiment, the proximal end of said cannula body comprises an attachment structure for removably coupling an aspiration source, such as a luer attachment structure. In a preferred embodiment, the proximal end of the device comprises viewable indicia, such as a marking indicating the position of the laterally oriented distal opening.

The invention also provides a bone biopsy system comprising:

an outer cannula;

a handle portion coupled to the proximal end of said outer cannula;

wherein said outer cannula is adapted to removably accommodate therein a biopsy aspiration device adapted to obtain a bone marrow sample comprising: an elongated cannula body having a proximal end, and a distal tip and a linear longitudinal axis;

a lumen running longitudinally through the interior of said cannula body, said lumen terminating at a proximal opening and terminating at a single laterally oriented distal opening immediately adjacent to the distal tip;

wherein said distal tip of said cannula body comprises an arcuate curved surface on the side opposite to said laterally oriented distal opening, said arcuate curved surface terminating at said distal opening.

In a preferred embodiment, the outer cannula is adapted for obtaining a core sample of bone tissue. Accordingly, the outer cannula can accommodate the biopsy aspiration device of the invention within, so as to permit sequential marrow and bone sampling at the same biopsy site.

The invention also provides a method of obtaining a bone marrow sample from a marrow sampling site in a patient comprising the steps of:

(a) penetrating the cortex of a bone with an outer cannula having a stylet positioned within, the distal portion of said stylet extending beyond the distal end of said outer cannula, until the distal end of said outer cannula is surrounded by marrow;

(b) removing said stylet from said outer cannula;

(c) inserting into said outer cannula a biopsy aspiration device such that the distal tip of said biopsy aspiration device is extended into marrow, said biopsy aspiration device comprising:

an elongated cannula body having a proximal end, a distal tip and a linear longitudinal axis;

a lumen running longitudinally through the interior of said cannula body, said lumen terminating at a proximal opening and terminating at a single laterally oriented distal opening immediately adjacent to the distal tip;

wherein said distal tip of said cannula body comprises an arcuate curved surface on the side opposite to said laterally oriented distal opening, said arcuate curved surface terminating at said distal opening;

(d) attaching an aspiration source to the proximal end of said biopsy aspiration device; and

(e) withdrawing a sample of marrow from a marrow sampling site.

### BRIEF DESCRIPTION OF THE DRAWINGS

FIG. 1 is an angled side view perspective of the biopsy aspiration device according to one embodiment of the invention.

FIG. 2 is a side view of the biopsy aspiration device with the distal opening facing upwards according to one embodiment of the invention.

FIG. 3 is a magnified side view of the distal portion of the biopsy aspiration device according to one embodiment of the invention.

FIG. 4 is a magnified frontal view of the distal portion of the biopsy aspiration device according to one embodiment of the invention.

FIG. 5 is a bone biopsy system containing the biopsy aspiration device positioned within and attached to an aspiration source according to one embodiment of the invention.

FIG. 6 is a cross-sectional schematic view of a bone containing the distal portions of a bone biopsy system with the biopsy aspiration device positioned within a cannula in accordance with one embodiment of the invention.

FIG. 7 is a sequential depiction of biopsy procedure using a bone biopsy system containing the biopsy aspiration device according to one embodiment of the invention.

FIG. 8 is a cross-sectional side view showing the interior positioning of the bone biopsy system components according to one embodiment of the invention.

### DETAILED DESCRIPTION OF THE INVENTION

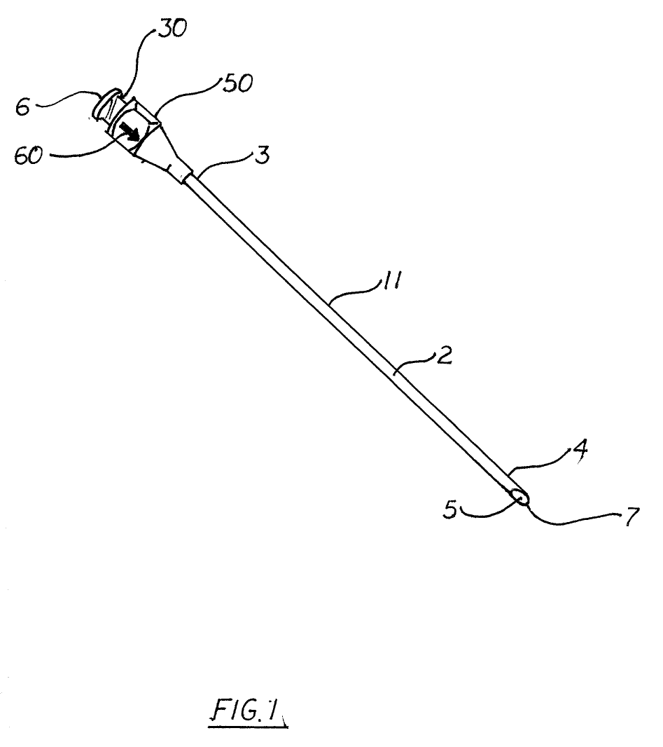

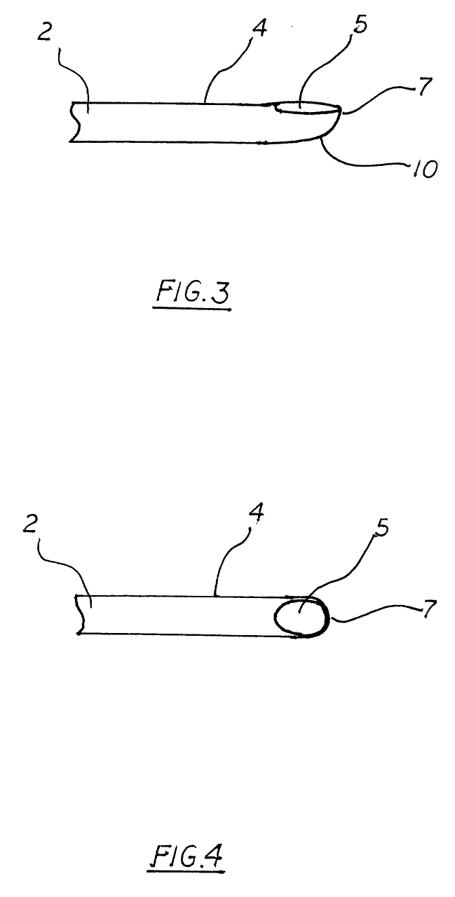

In general, the biopsy aspiration device of the invention is adapted to obtain a bone marrow sample. Referring to FIG. 1, the biopsy aspiration device comprises an elongated cannula body 2 having a proximal end 3 and distal end 4 and a linear longitudinal axis. A lumen runs longitudinally through the interior of the cannula body 2 and terminates at a proximal opening 6 and terminates at a single laterally oriented distal opening 5 immediately adjacent to the distal tip 7. The distal end 4 of the cannula body 2 comprises an arcuate curved surface 10 (see FIG. 3) on the side opposite to said laterally oriented distal opening 5. The arcuate curved surface 10 terminates at the distal opening 5.

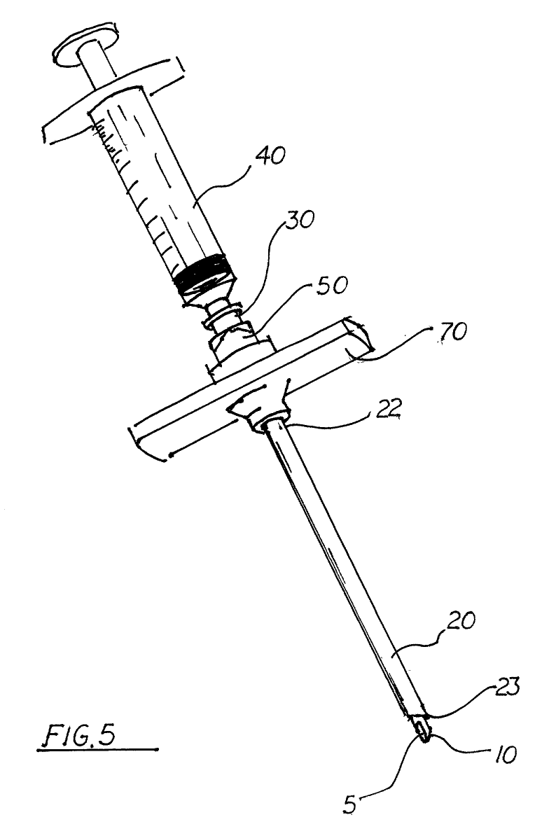

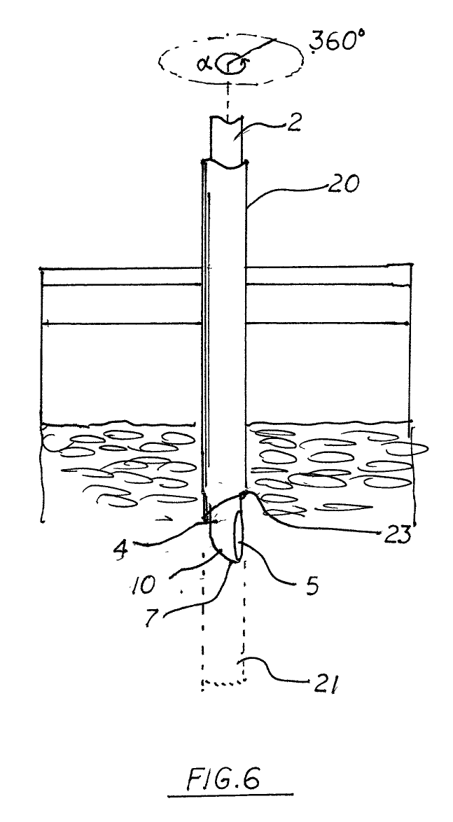

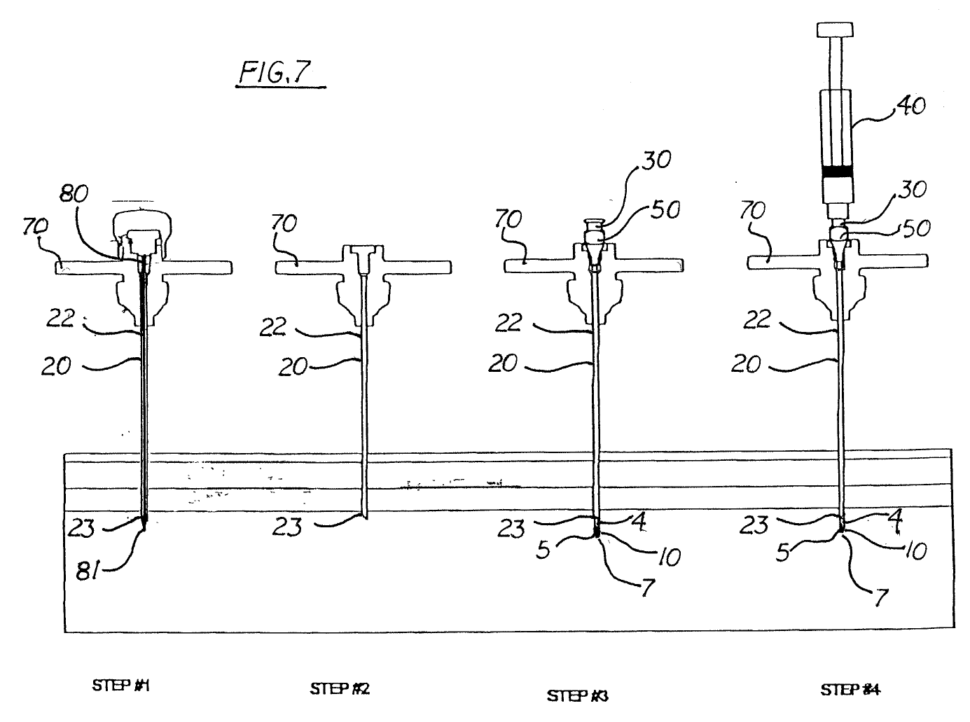

As can be seen from FIGS. 3 and 4, the arcuate curved surface 10 and laterally oriented distal opening 5 at the distal portion 4 of the device collectively form a spoon or scoop-like configuration. The distal portion 4 of the device is substantially linear along with the medial portion 11 of the cannula body 2, and therefore is adapted for removable insertion within and through the distal end 23 of an outer cannula 20 structure as shown in FIGS. 5 and 6. Accordingly, the arcuate curved surface 10 is substantially confined to the outer dimensions of the remaining portion of the cannula body 2 and the inner dimensions of the outer cannula 20.

Now referring to FIG. 6, as the biopsy aspiration device is advanced into the bone, the laterally oriented distal opening 5 is less likely to become blocked by the tissue since the tissue encountered by the cannula advancement in the longitudinal direction is diverted over the arcuate curved surface 10 of the cannula body 2. The arcuate curved shape further introduces the aspiration device to the tissue in a more gradual manner without a substantially compressive impact or “coring” effect of the tissue. Accordingly, the aspiration device of the invention reduces or avoids compromising the structural integrity of the bone tissue located distally to the aspiration device (represented by dotted line 21). The laterally oriented distal opening 5 aspires marrow residing in the tissue from a lateral direction thereby reducing the amount of marrow removed from the distal tissue. As a result, a core sample obtained by advancing an outer cannula in the longitudinal direction after removal of the biopsy aspiration device is less adulterated by the aspiration step.

Because the biopsy aspiration device of the invention can be rotated 360° to obtain marrow from the entire circumference surrounding the distal portion 4 of the device (rotational range represented as α in FIG. 6), the device can avoid tissue blockage or dry pockets which can be encountered during marrow withdrawal. This ensures that an adequate sample volume is obtained.

The cannula body 2 can be composed of any rigid material which can retain its structural integrity and configuration in response to the forces exerted upon it which are typically associated with bone biopsy procedures. Suitable materials which can be used for the cannula body include, but are not limited to, stainless steel or titanium.

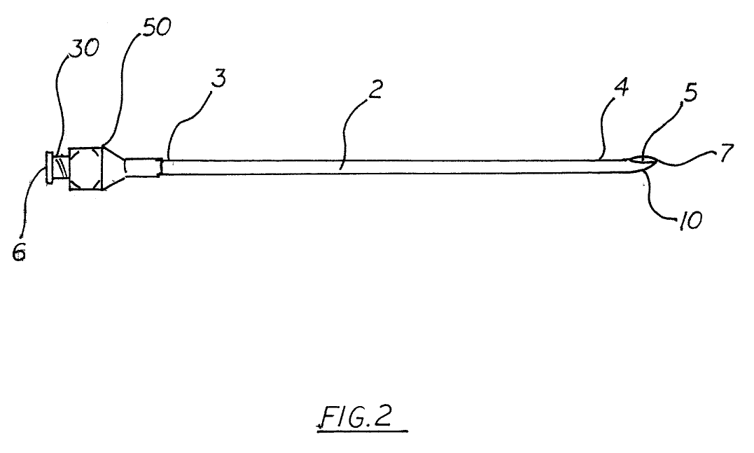

In a preferred embodiment and as depicted in FIGS. 2 and 5, the proximal end 3 of the cannula body 2 comprises an attachment structure 30 for removably coupling an aspiration source 40 (shown as a syringe for illustrative purposes) to the biopsy aspiration device of the invention. Any attachment structure 30 which can create a continuous fluid conduit when coupled to an aspiration source can be used. Suitable attachment structures 30 include, but are not limited to, a hub 50 containing an interfitting threaded luer structure as shown in the figures. When a hub 50 is used, the hub 50 surface can further comprise a surface structure which facilitates gripping and manipulation by the user.

Aspiration sources which can be used include any aspiration source having a component which can couple to the proximal end or attachment structure of the device and effect the withdrawal of marrow from bone. Suitable aspiration sources include, but are not limited to, vacuum sources and associated tubing, siphons, syringes (as shown in the figures), and the like.

In a more preferred embodiment, the attachment structure 30 at the proximal end of the device comprises a hub 50 including a viewable indicia, such as a marking 60 indicating the position of the laterally oriented distal opening. Accordingly, the user can continually monitor the position of the laterally oriented distal opening 5 throughout the procedure.

The biopsy aspiration cannula can be a component of a bone biopsy system. With reference to FIG. 5, a bone biopsy system can comprise an outer cannula 20 and a handle portion 70 coupled to the proximal end 22 of the outer cannula 20. The outer cannula 20 can be adapted to removably accommodate the biopsy aspiration device such that the aspiration device can be inserted through the interior of the outer cannula 20 and the distal portion 4 of the aspiration device can extend beyond the distal tip 23 of the outer cannula 20.

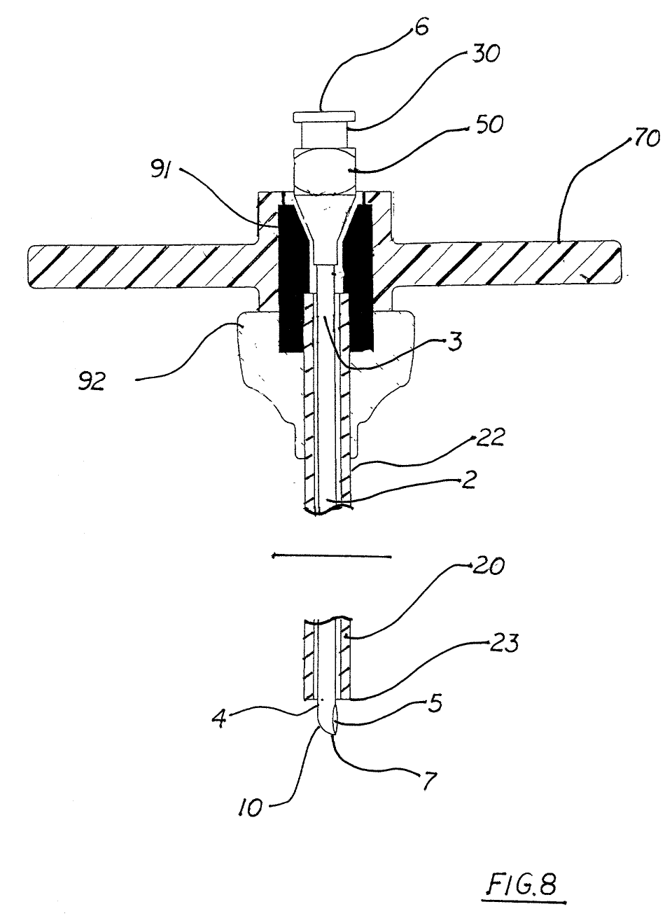

The bone biopsy system can also comprise a stylet 80 (see FIG. 7) adapted for removable insertion within the outer cannula 20. The stylet 80 can have a sharpened distal tip 81 such that when positioned within the outer cannula 20 the distal tip 81 of the stylet 80 extends beyond the distal tip 23 of the outer cannula 20 and thereby facilitates the penetration of the outer cannula 20 through tissue and bone.

In a preferred embodiment, the outer cannula 20 is adapted for obtaining a core sample of bone tissue. For example, the distal tip 23 of the outer cannula 20 can include a beveled or sharpened edge. Accordingly, an outer coring cannula can accommodate the biopsy aspiration device of the invention within so as to permit sequential marrow and bone sampling at the same biopsy site.

The various components of the biopsy aspiration device, whether metallic or plastic, can be made using conventional machining, molding and/or tooling equipment and techniques readily available in the art. For components which are irreversibly attached, various bonding, welding and fusing techniques which are conventional and readily available can be used.

Now referring to FIG. 8, one embodiment of a preferred bone biopsy system is illustrated. As seen from the figure, the outer cannula 20 is attached to an outer cannula hub 91, which receives or engages the distal portion of the hub 50 of the aspiration device. The outer cannula hub 91 can be secured in place on the handle portion 70 by an engagement structure, such as an end cap 92. The region of the handle portion 70 which interacts with the end cap 92 can be constructed to securely engage the end cap 92. Any suitable cooperative engaging structure can be used at this location.

Preferably, the juncture between the attachment structure 30 of the aspiration device (depicted as comprising a hub 50) and the outer cannula hub 91 creates a substantially air tight seal when the two components are engaged. Accordingly, the transport of air across the space between the aspiration cannula body 2 and the interior surface of the outer cannula 20 is minimized, thereby reducing or eliminating air from the exterior environment into the bone during aspiration of marrow. The hub-hub interface, therefore, can comprise interlocking features, such as a luer structure (not shown), and/or a sealing element, such as a gasket, o-ring, or the like. A preferred construction of the bone biopsy system of the invention comprises an outer cannula hub 91 composed of metal and an aspiration device hub 50 composed of plastic for the hub-hub interface. In one preferred embodiment, the bone biopsy system can comprise an outer cannula hub 91 and outer cannula 20 both composed of stainless steel and welded or fused together, and an aspiration device hub 50 composed of plastic, such as polycarbonate. Such a metal-to-plastic interface contributes to create a substantially air tight engagement at the hub-hub interface.

### EXAMPLE

### Bone Biopsy Procedure

The following is an example of a biopsy procedure in which a marrow and bone core sample are obtained at a single sampling site. The procedure is illustrated in FIG. 7.

A patient is prepared for a bone biopsy procedure in accordance with standard medical practice. The sampling site is selected by the practitioner. The bone biopsy system includes an outer cannula 20 coupled to a handle portion 70 at the proximal end 22, a stylet 80 and biopsy aspiration device. Initially and as shown in Step 1, the stylet 80 is inserted and secured within the outer cannula 20 such that the distal tip 81 of the stylet 80 extends beyond the distal tip 23 of the outer cannula 20. The tissue and cortex of the bone is penetrated by exerting force upon the handle portion 70 thereby advancing the outer cannula 20 with the stylet 80 further into the bone in the longitudinal direction. Once the softer trabecular bone region is penetrated by the distal portions of the outer cannula and stylet and the distal portions of the outer cannula and stylet are surrounded by marrow, the stylet 80 can be removed as shown in Step 2.

At this stage, an aspiration source can be attached to the attachment structure 30 of the biopsy aspiration device either before insertion of the device into the outer cannula 20 or after the aspiration device has been positioned within the outer cannula 20. In Step 3, the aspiration device is shown inserted into the outer cannula 20 so that the distal portion 4 of the aspiration device extends beyond the distal tip 23 of the outer cannula 20 and into the surrounding marrow. An aspiration source 40, such as a syringe, can be attached to the aspiration device as shown in Step 4. With the laterally oriented distal opening 5 of the aspiration device exposed to the marrow, aspiration can be effected by activation of the aspiration source to withdraw a marrow sample.

According to the invention, the biopsy aspiration device can be rotated 360° during the marrow sampling step. Thus, not only can the aspiration device ensure an adequate volume and comprehensive sampling profile, but can avoid obstruction of marrow intake from the surrounding tissue or inadequate marrow from dry pockets.

When an outer coring cannula is used as a component of the bone biopsy system, after the marrow sample is obtained the biopsy aspiration device can be withdrawn from the outer cannula. The outer cannula can then advanced further into the bone to obtain a core sample.

One advantage associated with the invention is that because the aspiration device withdraws marrow from the lateral direction, the marrow sample obtained is less adulterated by the damaged bone distal to the tip. Because the marrow is obtained laterally and the distal tip of the aspiration device is arcuate or curved, the damage to the tissue located distally to the aspiration device in and of itself is minimized. Using the invention, a core sample can be obtained at the same sampling site which also includes marrow. Accordingly, the core sample exhibits a more accurate natural composition of the patient's tissue. In summary, improved marrow samples and core samples can be obtained from a single sampling site without the need for multiple penetration of the patient's bone.

### INDUSTRIAL APPLICABILITY

The invention is useful in bone biopsy procedures where collection of a sample of bone marrow is needed. The invention can be used in conjunction with bone core biopsy sampling techniques and equipment, wherein obtaining both a marrow sample and core sample at a single sampling site is preferred. Since the number of sampling sites can be reduced, patient discomfort can be reduced.

This invention has been described with reference to various specific and preferred embodiments and techniques. It will be understood, however, that reasonable variations and modifications of such embodiments and techniques are possible without departing from the spirit and scope of the invention.

What is claimed is:

1. A biopsy aspiration device adapted to obtain a bone marrow sample comprising:
an elongated cannula body having a proximal end, a distal tip and a linear longitudinal axis;
a lumen running longitudinally through the interior of said cannula body, said lumen terminating at a proximal opening and terminating at a single laterally oriented distal opening immediately adjacent to the distal tip;
wherein said distal tip of said cannula body comprises an arcuate curved surface originating on the opposite side to said laterally oriented distal opening, said arcuate curved surface terminating at the distal-most point of said distal opening.

2. The biopsy aspiration device according to claim 1 wherein said proximal end of said cannula body further comprises an attachment structure for removably coupling an aspiration source.

3. The biopsy aspiration device according to claim 2 wherein said attachment structure comprises a luer attachment.

4. The biopsy aspiration device according to claim 1 wherein the proximal end of the device comprises viewable indicia.

5. The biopsy aspiration device according to claim 4 wherein said viewable indicia comprises a marking indicating the position of the laterally oriented distal opening.

6. A bone biopsy system comprising:
an outer cannula;
a handle portion coupled to the proximal end of said outer cannula;
wherein said outer cannula is adapted to removably accommodate therein a biopsy aspiration device, said biopsy aspiration device being adapted to obtain a bone marrow sample and comprising:
an elongated cannula body having a proximal end, a distal tip and a linear longitudinal axis;
a lumen running longitudinally through the interior of said cannula body, said lumen terminating at a proximal opening and terminating at a single laterally oriented distal opening immediately adjacent to the distal tip;
wherein said distal tip of said cannula body comprises an arcuate curved surface on the side opposite side to said laterally oriented distal opening, said arcuate curved surface terminating at said distal opening.

7. The bone biopsy system according to claim 6, wherein said proximal end of the cannula body of said biopsy aspiration device further comprises an attachment structure for removably coupling an aspiration source.

8. The bone biopsy system according to claim 7 wherein said attachment structure of comprises a luer attachment.

9. The bone biopsy system according to claim 6 wherein the proximal end of said biopsy aspiration device comprises viewable indicia.

10. The bone biopsy system according to claim 9 wherein said viewable indicia comprises a marking indicating the position of the laterally oriented distal opening.

11. The bone biopsy system according to claim 6 further comprising a stylet adapted for removable insertion within said outer cannula.

12. A method of obtaining a bone marrow sample from a marrow sampling site in a patient comprising the steps of:
(a) penetrating the cortex of a bone with an outer cannula having a stylet positioned within, the distal portion of said stylet extending beyond the distal end of said outer cannula, until the distal end of said outer cannula is surrounded by marrow;
(b) removing said stylet from said outer cannula;
(c) inserting into said outer cannula a biopsy aspiration device such that the distal tip of said biopsy aspiration device is extended into marrow, said biopsy aspiration device comprising:
an elongated cannula body having a proximal end, a distal tip, and a linear longitudinal axis;
a lumen running longitudinally through the interior of said cannula body, said lumen terminating at a proximal opening and terminating at a single laterally oriented distal opening immediately adjacent to the distal tip;
wherein said distal tip of said cannula body comprises an arcuate curved surface on the side opposite side to said laterally oriented distal opening, said arcuate curved surface terminating at said distal opening;
(d) attaching an aspiration source to the proximal end of said biopsy aspiration device; and
(e) withdrawing a sample of marrow from a marrow sampling site.

13. The method according to claim 12 further comprising the step of rotating said biopsy aspiration device within said outer cannula thereby repositioning the laterally oriented distal opening within the marrow sampling site.

14. The method according to claim 12 further comprising:
(f) removing the biopsy aspiration device from the outer cannula; and
(g) further advancing the outer cannula into the bone to obtain a core sample.
